(19) United States
(12) Patent Application Publication
(10) Pub. No.: US 2025/0367220 A1
(43) Pub. Date: Dec. 4, 2025

(54) COMPOSITIONS COMPRISING CANNABINOIDS AND METHODS OF PREPARATION AND USE THEREOF

(71) Applicant: New Growth Brands, Inc., San Diego, CA (US)

(72) Inventors: Roy LIPSKI, San Diego, CA (US); James STEPHENS, San Diego, CA (US)

(73) Assignee: New Growth Brands, Inc., San Diego, CA (US)

(21) Appl. No.: 18/874,811

(22) PCT Filed: Jun. 16, 2023
(86) PCT No.: PCT/US2023/068615

§ 371 Date: Dec. 13, 2024

Related U.S. Application Data
(60) Provisional application No. 63/353,250, filed on Jun. 17, 2022.

(51) Int. Cl. A61K 31/00; A61K 9/00; A61K 36/185; A61K 47/14

(52) U.S. Cl. CPC A61K 31/658; A61K 9/007; A61K 9/0095; A61K 36/3482; A61K 36/577; A61K 47/14

(57) ABSTRACT

Compositions that include cannabinoids other than tetrahydrocannabinol (THC), such as cannabigerol (CBG) in combination with tetrahydrocannabinol (THC) or formulated to be combined with THC, and methods of preparation and use thereof are described. The compositions may be useful to reduce the risk of THC-induced anxiety and/or to produce a different intoxicating effect compared to THC alone.

### CROSS-REFERENCE TO RELATED APPLICATIONS

This application claims priority to U.S. Provisional Application No. 63/353,250, filed on Jun. 17, 2022, which is incorporated by reference herein in its entirety.

### TECHNICAL FIELD

The present disclosure relates generally to compositions comprising cannabinoids, including compositions comprising cannabinoids other than tetrahydrocannabinol (THC), such as cannabigerol (CBG) in combination with tetrahydrocannabinol (THC) or formulated to be combined with THC and methods of preparation and use thereof. The compositions may be used to reduce the risk of THC-induced anxiety and produce a different intoxicating effect compared to THC alone.

### BACKGROUND

While many users enjoy consuming various cannabis products, some report adverse effects and/or are unsatisfied by products currently available. For example, some users have reported anxiety or paranoia associated with cannabis, usually associated with the presence of THC. Other users would prefer lower amounts of THC but still seek beneficial effects of consuming cannabis.

### SUMMARY

The present disclosure includes compositions useful for reducing one or more drawbacks of current cannabis/THC-containing products and/or improving overall user experience of consuming such products. For example, the present disclosure includes a composition comprising tetrahydrocannabinol (THC) or tetrahydrocannabinolic acid (THCA) and cannabigerol (CBG) or cannabigerolic acid (CBGA) in a weight ratio THC or THCA:CBG or CBGA ranging from about 1:10 to about 10:1, such as about 1:5 to about 5:1, about 1:3 to about 3:1, about 1:2 to about 2:1, or about 1:1. The composition may be formulated as a liquid, a waxy material, a gel, or a solid. In some examples, the composition further comprises at least one terpene, wherein the composition comprises less than or equal to 10% by weight terpenes, based on the total weight of the composition. The composition may further comprise at least one terpene chosen from α-pinene, β-pinene, limonene, β-myrcene, β-caryophyllene, linalool, valencene, humulene, myrcene, bisabolol, geraniol, humulene, farnesene, fenchol, terpineol, borneol, nerolidol, (-)-caryophyllene oxide, terpinolene, or combinations thereof.

In some examples, the composition comprises at least 5 mg CBG or CBGA per 0.125-100 g of the composition. The composition may further comprise one or more cannabinoids other than CBG, CBGA, THC, and THCA. For example, the one or more cannabinoids other than CBG, CBGA, THC, and THCA may be selected from cannabidiol (CBD), cannabichromene (CBC), cannabicyclol (CBL), cannabivarin (CBV), cannabielsoin (CBE), cannabicitran (CBT), cannabigerovarin (CBGV), tetrahydrocannabivarin (THCV), cannnabidivarin (CBDV), cannabinol (CBN), acidic forms thereof, or any combination thereof. In at least one example, the composition comprises less than 1% by weight CBD or acidic form thereof, based on the total weight of the composition.

According to some aspects, the composition may be formulated as a smokeable vaping liquid, a smokeable waxy material, or a smokeable herbal material. For example, the composition may be formulated as a smokeable herbal material and (a) does not comprise marijuana, (b) does not comprise hemp, or (c) does not comprise marijuana or hemp. In some examples, the composition may be formulated as a pharmaceutical, a food, or a beverage. The composition may be used for reducing the risk of THC-induced anxiety and/or producing a different intoxicating effect compared to THC alone.

The present application also includes a method of reducing the risk of THC-induced anxiety and/or producing a different intoxicating effect compared to THC alone, the method comprising ingesting and/or inhaling a composition as described above and/or elsewhere herein. Another aspect of the present disclosure includes a method of providing the same or similar level of potency as compared to a reference composition with a greater amount of THC or THCA per gram of the composition, the method comprising ingesting and/or inhaling a composition as described above and/or elsewhere herein. For example, the composition may be in the form of a pharmaceutical, a food, a beverage, smoke, or vapor from a vaping device or e-cigarette. In another aspect, the present application includes a method of reducing the risk of THC-induced anxiety and/or producing a different intoxicating effect compared to THC alone, comprising administering to a subject a composition as described above and/or elsewhere herein. The composition may be in the form of a pharmaceutical, a food, a beverage, smoke, or vapor from a vaping device or e-cigarette.

Also disclosed herein is a method of altering an intoxicating experience of cannabis or tetrahydrocannabinol (THC), the method comprising inhaling smoke or vapor from a smokeable material. The smokeable material may comprise cannabis or other THC-containing material and a composition comprising one or more smokeable herbs that are not marijuana and one or more cannabinoids that are not tetrahydrocannabinol (THC) or tetrahydrocannabinolic acid (THCA), wherein the composition comprises less than 0.3% by weight THC and THCA, based on the total weight of the composition. The present application further includes a method of preparing a smokeable material, the method comprising combining a composition with cannabis or other THC-containing material, wherein the composition comprises one or more smokeable herbs that are not marijuana, and one or more cannabinoids that are not tetrahydrocannabinol (THC) or tetrahydrocannabinolic acid (THCA), wherein the composition comprises less than 0.3% by weight THC and THCA, based on the total weight of the composition.

In some examples herein, the composition further comprises one or more terpernes. For example, the one or more terpenes may be chosen from α-pinene, β-pinene, limonene, β-myrcene, β-caryophyllene, linalool, valencene, humulene, myrcene, bisabolol, geraniol, humulene, farnesene, fenchol, terpineol, borneol, nerolidol, (-)-caryophyllene oxide, terpinolene, and combinations thereof. In some aspects, the one or more cannabinoids that are not THC or THCA may include cannabigerol (CBG) or cannabigerolic acid (CBGA). Additionally or alternatively, the one or more cannabinoids that are not THC or THCA may include cannabidiol (CBD), cannabichromene (CBC), cannabicyclol (CBL), cannabivarin (CBV), cannabielsoin (CBE), cannabicitran (CBT), cannabigerovarin (CBGV), tetrahydrocannabivarin (THCV), cannnabidivarin (CBDV), cannabinol (CBN), acidic forms thereof, or any combination thereof.

Further disclosed herein is a composition comprising 0-10% by weight terpenes, 2-40% by weight cannabinoids that are not tetrahydrocannabinol (THC) or tetrahydrocannabinolic acid (THCA), and 50-98% by weight of at least one smokeable herb that is not marijuana, wherein the composition comprises less than 0.3% by weight THC and THCA, based on the total weight of the composition. The composition may comprise at least 5 mg of the cannabinoids that are not THC or THCA per 0.125-100 g of the composition. In some aspects, the cannabinoids that are not THC or THCA may include cannabigerol (CBG) or cannabigerolic acid (CBGA). Additionally or alternatively, the one or more cannabinoids that are not THC or THCA may include cannabidiol (CBD), cannabichromene (CBC), cannabicyclol (CBL), cannabivarin (CBV), cannabielsoin (CBE), cannabicitran (CBT), cannabigerovarin (CBGV), tetrahydrocannabivarin (THCV), cannnabidivarin (CBDV), cannabinol (CBN), acidic forms thereof, or any combination thereof. The composition may comprise less than 1% by weight CBD or acidic form thereof, based on the total weight of the composition.

The smokeable herb may comprise Althaea officinalis L, Amaranthus dubius, Arctostaphylos uva-ursi, Argemone mexicana, Arnica, Artemisia vulgaris, Asteraceae, Brassica oleracea, Calea zacatechichi, Canavalia maritima, Cecropia obtusifolia, Cestrum nocturnum, Cynoglossum virginianum, Cytisus scoparius, Entada rheedii, Eschscholzia californica, Fittonia albivenis, Humulus japonicus, Lavandula, Lactuca virosa, Lamiaceae, Salvia, Scutellaria, Leonotis leonurus, Leonurus cardiaca, Leonurus sibiricus, Lobelia cardinalis, Lobelia inflata, Lobelia siphilitica, Nepeta cataria, Nicotiana, Nymphaea alba, Nymphaea caerulea, Papaver somnferum, Origanum majorana, Origanum vulgare, Passiflora incamata, Pedicularis densiflora, Pedicularis groenlandica, Rubus idaeus, Rubus occidentalis, Salvia divinorum, Salvia dorrii, Scutellaria galericulata, Scutellaria lateriflora, Scutellaria nana, Sida acuta, Sida rhombifolia, Silene capensis, Syzygium aromaticum, Tagetes lucida, Tarchonanthus camphoratus, Turnera diffusa, Tussilago farfara, Verbascum, Zornia latifolia, or mixtures thereof. In some examples, the composition further comprises at least one terpene, wherein the composition comprises less than or equal to 10% by weight terpenes, based on the total weight of the composition. In at least one example, the composition may further comprise at least one terpene chosen from α-pinene, β-pinene, limonene, β-myrcene, β-caryophyllene, linalool, valencene, humulene, myrcene, bisabolol, geraniol, humulene, farnesene, fenchol, terpineol, borneol, nerolidol, (-)-caryophyllene oxide, terpinolene, or combinations thereof.

The composition(s) as described above and/or elsewhere herein may be used for reducing the risk of THC-induced anxiety and/or producing a different intoxicating effect compared to THC alone when consumed with THC, cannabis, or other THC-containing material. The present disclosure also includes a smokeable material comprising a composition as described above and THC, cannabis, or other THC-containing material. Further disclosed herein is a method of preparing the smokeable material, the method comprising combining a composition as described above with THC, cannabis, or other THC-containing material. The smokeable material may be used for reducing the risk of THC-induced anxiety and/or producing a different intoxicating effect compared to THC alone.

Also disclosed herein is a method of reducing the risk of THC-induced anxiety and/or producing a different intoxicating effect compared to THC alone, the method comprising inhaling smoke or vapor from a smokeable material as described above and/or elsewhere herein or administering the smokeable material to a subject.

### DETAILED DESCRIPTION

Particular aspects of the present disclosure are described in greater detail below. The terms and definitions provided herein control, if in conflict with terms and/or definitions incorporated by reference.

As used herein, the terms “comprises,” “comprising,” or any other variation thereof are intended to cover a non-exclusive inclusion, such that a process, method, composition, article, or apparatus that comprises a list of elements does not include only those elements, but may include other elements not expressly listed or inherent to such process, method, composition, article, or apparatus. The term “exemplary” is used in the sense of “example” rather than “ideal.” The singular forms “a,” “an,” and “the” include plural reference unless the context dictates otherwise. The terms “approximately” and “about” refer to being nearly the same as a referenced number or value. As used herein, the terms “approximately” and “about” generally should be understood to encompass ±5% of a specified amount or value.

The present disclosure includes compositions comprising cannabigerol (CBG) in combination with tetrahydrocannabinol (THC), and compositions comprising cannabinoids other than THC formulated to be combined with THC in use. The cannabinoids of the compositions herein may be extracted (e.g., isolated, distilled, etc.) from a natural cannabis source, such as cannabis plant, and optionally purified, or created via chemical or biological synthesis. Cannabigerolic acid (CBGA) and tetrahydrocannabinolic acid (THCA) decarboxylate to CBG and THC respectively, e.g., when heated. In the present disclosure, all references to CGB include CGBA, and all references to THC include THCA. Reference to cannabinoids herein includes all isomers including enantiomers thereof.

Without being bound by theory, it is believed that CBG, which is a non-psychoactive cannabinoid, and THC, which is psychoactive, have a synergistic effect that, when consumed (e.g., inhaled) by a user provides a different effect on the user than THC alone. For example, synergy provided by CBG and THC according to the present disclosure may reduce, alleviate, or prevent anxiety or paranoia associated with THC consumption (e.g., THC-induced anxiety and/or paranoia). Symptoms of anxiety associated with THC consumption in some users may include, but are not limited to, increased heart rate, racing thoughts, sweating, shaking, chest pain, nausea, stomach pain, choking sensations, intense feelings of fear, difficulty breathing, and feelings of impending doom. Further, for example, synergy provided by CBG and THC according to the present disclosure may improve user experience, e.g., providing a better user experience as compared to THC alone. In some examples, synergy provided by CBG and THC according to the present disclosure may provide the same or similar level of potency experienced by a user but with lower amounts of THC as compared to a composition with higher amounts of THC but without CBG. Compositions according to the present disclosure may provide a more relaxed (e.g., mellow or happy) consumption experience as compared to THC alone.

### Compositions with THC and CBG

The compositions herein may comprise CBG and THC, e.g., in the form of a solid (including, e.g., a leafy herb/plant material, and edible foods and confectionaries), liquid (including, e.g., an emulsion), waxy material, or gel. The composition may comprise higher proportions of CBG as compared to the content of CBG in natural cannabis.

In some examples, the composition comprises THC and CBG in a weight ratio (THC:CBG) ranging from about 1:10 to about 10:1, such as from about 1:9 to about 9:1, from about 1:8 to about 8:1, from about 1:7 to about 7:1, from about 1:6 to about 6:1, from about 1:5 to about 5:1, from about 1:4 to about 4:1, from about 1:3 to about 3:1, from about 1:2 to about 2:1, or about 1:1. For example, the composition may comprise THC and CBG in a weight ratio (THC:CBG) ranging from about 1:1 to about 2.4:1. In at least one example, increasing the amount of CBG in the composition relative to the amount of THC, such that the amount of CBG is greater than the amount of THC, may provide for a better user experience as compared to THC alone as discussed in further detail below. The composition may comprise at least 5 mg CBG per serving, wherein a typical serving may from range from about 0.125-100 g. For example, the composition may comprise at least 7.5 mg, at least 10 mg, at least 12 mg, at least 15 mg, at least 20 mg, at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg, or at least 100 mg per serving.

Additionally or alternatively to CBG, the compositions herein may comprise one or more other cannabinoids (and/or their acidic forms) other than THC such as, e.g., cannabidiol (CBD), cannabichromene (CBC), cannabicyclol (CBL), cannabivarin (CBV), cannabielsoin (CBE), cannabicitran (CBT), cannabigerovarinic acid (CBGVA), cannabigerovarin (CBGV), tetrahydrocannabivarin (THCV), cannnabidivarin (CBDV) and/or cannabinol (CBN). In some examples, the composition comprises CBG and THC but comprises less than 1% by weight CBD.

The compositions herein may comprise one or more terpenes and/or other flavors. Exemplary flavors include, but are not limited to, tobacco, fruit, spice, herb, alcohol, candy, menthol, and vanilla. Exemplary terpenes include, but are not limited to, α-pinene, β-pinene, limonene, β-myrcene, β-caryophyllene, linalool, valencene, humulene, myrcene, bisabolol, geraniol, humulene, farnesene, fenchol, terpineol, borneol, nerolidol, (-)-caryophyllene oxide, and terpinolene. In some examples, the composition comprises 0-10% by weight terpenes, compared to the total weight of the composition. For example, the composition may comprise 1-5% by weight, 3-7% by weight, or 6-10% by weight terpenes. In some examples, the composition comprises less than 5% by weight, less than 3% by weight, less than 1% by weight, or less than 0.5% by weight terpenes.

The compositions comprising CBG and THC may be prepared using various techniques, such as mixing or blending. In some examples, the CBG and THC may be combined with carrier liquid(s) such as water, alcohol, and/or oil or other type of carrier, such as a waxy material. In at least one example, the composition comprises CBG and THC in combination with water and oil to form an emulsion. Optionally, one or more emulsifiers may also be added to the emulsion. Exemplary carrier oils that may be used for the compositions here include, but are not limited to, medium-chain triglyceride (MCT) oil, 8-carbon fatty acids, 9-carbon fatty acids, and 10-carbon fatty acids.

Such compositions comprising CBG and THC may be added to an edible material or product, such as a food or beverage. Food/beverage additives such as flavoring agents, sweeteners, preservatives, and/or antioxidants may also be added. In some examples, the composition may comprise a pharmaceutical.

Additionally or alternatively, such compositions comprising CBG and THC may be combined with an herbal material or other plant material to thereby infuse and/or coat the herb/plant with the CBG and THC. Exemplary herbal materials and other plant materials suitable for the present disclosure include, but are not limited to, Althaea officinalis L (marshmallow leaf), Amaranthus dubius, Arctostaphylos uva-ursi (bearberry), Argemone mexicana, Arnica, Artemisia vulgaris (mugwort), Asteraceae (chamomile), Brassica oleracea (cabbage), Calea zacatechichi, Cannabis sativa (cannabis/marijuana or hemp), Canavalia maritima (baybean), Cecropia obtusifolia, Cestrum nocturnum, Cynoglossum virginianum (wild comfrey), Cytisus scoparius, Entada rheedii, Eschscholzia californica (California poppy), Fittonia albivenis, Humulus japonicus (Japanese hops), Lavandula (lavender),Lactuca virosa (lettuce opium), Lamiaceae (mint), Salvia (sage), Scutellaria (skullcap), Leonotis leonurus (lion's tail), Leonurus cardiaca (motherwort), Leonurus sibiricus (honeyweed), Lobelia cardinalis, Lobelia inflata (Indian tobacco), Lobelia siphilitica, Nepeta cataria (catnip), Nicotiana, Nymphaea alba (white lily), Nymphaea caerulea (blue lily), Papaver somnferum (opium poppy), Origanum majorana (marjoram), Origanum vulgare (oregano), Passiflora incarnata (passionflower), Pedicularis densiflora (Indian warrior), Pedicularis groenlandica (elephant's head), Rubus idaeus (red raspberry), Rubus occidentalis, Salvia divinorum, Salvia dorrii (tobacco sage), Scutellaria galericulata, Scutellaria lateriflora, Scutellaria nana, Sida acuta and/or Sida rhombifolia (wireweed), Silene capensis, Syzygium aromaticum (clove), Tagetes lucida (Mexican tarragon), Tarchonanthus camphoratus, Turnera diffusa (damiana), Tussilago farfara (coltsfoot), Verbascum (mullein), Zornia latifolia (maconha brava), or mixtures thereof. Some hemp materials, such as Althaea officinalis L (marshmallow leaf) may reduce or eliminate coughing or other symptoms caused by throat irritation. For example, marshmallow leaf may serve as a tobacco substitute and may aid in providing a smoother smoking experience (e.g., less harsh, less coughing). Cannabis sativa includes both marijuana and hemp, wherein hemp generally comprises less than 0.3% by weight THC and THCA, and marijuana generally comprises greater than 0.3% by weight THC and THCA in the buds of the plant. In some examples herein, the composition comprises Cannabis sativa in the form of hemp and does not comprise marijuana. In some examples, the composition comprises Cannabis sativa in the form of marijuana and does not comprise hemp. In some examples, the composition comprises Cannabis sativa in the form of both marijuana and hemp.

The compositions herein may be formulated as a smokeable vaping liquid or smokeable material, e.g., a smokeable waxy material or herbal material. In some examples, the composition is formulated as smokeable herbal material and does not comprise marijuana, does not comprise hemp, or does not comprise marijuana or hemp.

### Smokeable Materials

Other compositions herein do not include THC but are formulated to be combined with THC in use. Such compositions may be free from (devoid of) THC. For example, the composition may comprise a smokeable material that does not comprise THC (or that comprises less than 1% by weight, less than 0.5% by weight, less than 0.3% by weight THC, or less than 0.1% by weight THC) but that can be combined with cannabis (including cannabis leaf, stem, and/or flower), THC, or THC-containing material to be smoked or vaped by a user. In use, the compositions comprising cannabinoids other than THC may be combined with cannabis, THC, or THC-containing material to form a smokeable material.

A smokeable material refers to any material that is capable of being combusted or vaporized and subsequently inhaled by a user. Exemplary smokeable materials include, but are not limited to, waxy materials, herbs and plants other than marijuana (including the exemplary herbal and plant materials listed above other than Cannabis sativa in the form of marijuana), vaporizable liquids, and waxy materials. Such compositions may comprise one or more cannabinoids other than THC such as, e.g., CBG (including CBGA), CBD, CBC, CBL, CBV, CBE, CBT, CBGV, THCV, CBDV, and/or CBN, as well as their corresponding acidic forms.

The compositions useful in or as smokeable materials may comprise about 2-40% by weight cannabinoids other than THC, based on the total weight to the composition, such as about 2-35% by weight, about 7-35% by weight, about 10-35% by weight, about 15-35% by weight, about 20-35% by weight, about 25-35% by weight, about 2-15% by weight, about 10-15% by weight, about 30-40% by weight, or about 12-25% by weight. In at least one example, the composition comprises less than 1% by weight CBD, such as less than about 0.5% or less than about 0.3% by weight CBD. In at least one example, composition does not comprise THC or CBD. Optionally, the composition may comprise one or more terpenes and/or one or more flavors. Exemplary flavors include, but are not limited to, tobacco, fruit, spice, herb, alcohol, candy, menthol, and vanilla. Exemplary terpenes include, but are not limited to, α-pinene, β-pinene, limonene, β-myrcene, β-caryophyllene, linalool, valencene, humulene, myrcene, bisabolol, geraniol, humulene, farnesene, fenchol, terpineol, borneol, nerolidol, (-)-caryophyllene oxide, and terpinolene. In some examples, the composition comprises 0-10% by weight terpenes, compared to the total weight of the composition. For example, the composition may comprise 1-5% by weight, 1-7% by weight, 1-3% by weight, or 2-10% by weight terpenes. In some examples, the composition comprises less than 5% by weight, less than 3% by weight, less than 1% by weight, or less than 0.5% by weight terpenes. In at least one example, the composition may be free from (devoid of) tobacco and/or nicotine.

The compositions comprising cannabinoids other than THC may be prepared using various techniques. In some examples, the cannabinoids (optionally in combination with terpenes and/or other additives or components) may be combined with a liquid or waxy carrier to provide a tincture, or may be combined with an herbal or plant material (including the examples listed above) to thereby infuse and/or coat the herb/plant material(s) with the cannabinoids other than THC. Exemplary waxy materials useful as carriers include, but are not limited to, hempwax, beeswax, paraffin wax, soy wax, mineral wax, and carnauba wax.

In at least one example, the composition comprises 0-10% by weight of one or more terpenes, 2-40% by weight cannabinoids that are not THC, and 50-98% by weight smokeable herb(s) that are not marijuana. In at least one example, the composition comprises about 1-5% by weight of one or more terpenes, about 5-35% by weight CBG, and about 60-94% by weight of one or more smokeable herbs that are not marijuana.

### Uses and Methods

As mentioned above, the compositions herein may be ingested or inhaled by a user. When ingested or inhaled, the composition may reduce, alleviate, or prevent a risk of THC-induced anxiety, including THC-induced anxiety symptoms (e.g., paranoia). In some examples, the composition may provide a dose of at least 5 mg CBG, e.g., wherein the total amount of product consumed or inhaled ranges from about 0.125-100 g. The compositions herein may improve user experience.

For example, a user may experience a better effect on mood upon ingesting or inhaling the compositions herein (e.g., compositions comprising CGB) as compared to compositions comprising THC alone. In some examples, the compositions herein may provide the same or similar intoxicating effect on a user with less THC, e.g., half the amount of THC. For example, the composition may comprise CBG and may reduce the amount of THC sufficient to provide a desired intoxicating effect.

The compositions herein may be used for reducing the risk of THC-induced anxiety and/or producing a different intoxicating effect compared to THC alone. An exemplary method according to the present disclosure includes reducing the risk of THC-induced anxiety and/or producing a different intoxicating effect compared to THC alone, the method comprising ingesting a composition as disclosed herein or administering to a subject a composition as disclosed herein. Examples of a different intoxicating effect provided by using the composition of the present disclosure as compared to using THC alone may include decreased feelings of anxiety, increased relaxation (e.g., zen), increased euphoria, better focus, sociability, and/or a more predictable experience during consumption. In contrast, using THC alone may result in anxiety, hallucinations, dysphoria, social withdrawal, impaired judgment and an unpredictable consumption experience. Another exemplary method according to the present disclosure includes providing the same or similar level of potency as compared to a reference composition with a greater amount of THC or THCA per gram of the composition, the method comprising ingesting the composition or administering to a subject a composition as disclosed herein. Such compositions may be ingested, for example, in the form of a pharmaceutical, a food, a beverage, smoke (e.g., from a combustible cigarette), or vapor from a vaping device or e-cigarette.

According to some examples herein, the present disclosure provides a method of altering an intoxicating experience of cannabis or THC, the method comprising inhaling smoke or vapor from a smokeable material, wherein the smokeable material comprises cannabis or other THC-containing material; and a composition comprising one or more smokeable herbs that are not marijuana and one or more cannabinoids that are not THC or THCA. The composition may comprise, for example, less than 0.3% by weight THC and THCA, based on the total weight of the composition.

For compositions herein that do not comprise THC or THCA (or that comprise less than 0.3% by weight, for example, of THC and THCA), such compositions may be used for reducing the risk of THC-induced anxiety and/or producing a different intoxicating effect compared to THC alone when consumed with THC, cannabis, or other THC-containing material.

### EXAMPLES

The following examples are intended to illustrate the present disclosure without, however, being limiting in nature. It is understood that the present disclosure encompasses additional embodiments consistent with the foregoing description and following examples.

### Example 1

A 0.5 gram amount of THC distillate (>95% pure by weight) was blended with 0.5 gram of CBG (>95% pure by weight), and then blended with 0.74 grams of HLB 14 emulsifier (non-ionic surfactant) that has been added to 1.1 grams of MCT (medium-chain triglycerides). This mixture was fully homogenized. Sixteen grams of water was added and the entire mixture was processed 150 cycles through a high-shear homogenization. The resultant emulsion base contained approximately 500 mg of THC and 500 mg of CBG with a target particle size of 300 nM. This emulsion base was added to lemonade at a ratio to create 10 servings, each serving containing 10 mg of THC and 10 mg of CBG.

Using the same ratios of cannabinoids to reagents and the same process as above, lemonade containing 10 servings of 10 mg of THC alone (no CBG) was produced.

### Study #1

An amount of 1000 units of 100 mg THC lemonade and 1000 units of 50 mg THC plus 50 mg of CBG lemonade were prepared as above. These were distributed to volunteer testers labelled as A and B. No information on which was THC only and which had THC and CBG was disclosed.

A group of 657 participants (348 female, 309 male) responded to a detailed questionnaire after sampling both drinks in separate sittings. Each could consume as much of the drink as they wished. Key findings were: 613 (93%) reported a positive experience or preferred the THC:CBG beverage compared to THC alone; and 501 (76%) reported that they felt the products were of the “same potency even if different”.

### Study #2

In order to investigate the relative effects with a single dose, 400 units of 5 mg THC and 400 units of 2.5 mg THC and 2.5 mg CBG were produced per the above. These were distributed to volunteer testers labelled as A and B. No information on which was THC only and which had THC and CBG was disclosed.

A group of 113 participants (79 female, 34 male) responded to a detailed questionnaire after sampling both drinks in separate sittings, consuming the entire beverage each time. Key findings were: 109 (96%) reported preferring the THC:CBG product over the THC-only product; 113 (100%) reported experiencing no anxiety or paranoia with the THC:CBG beverage, whereas 27 (24%) reported experiencing anxiety or paranoia with the THC-only beverage.

### Example 2

### Materials

Cannabis flower: Jack Herer reported to contain 24% THC. Infused marshmallow leaf: contained 85% dried, precut and sieved marshmallow leaf (Althaea officinalis L); 15% CBG; 5% terpenes. Terpenes comprised a Superlemon Haze flavor. CBG was a hemp extracted isolate containing 95% CBG by weight.

### Preparation

Approximately 7.5g of cannabis flower was blended with approximately 7.5g of infused marshmallow leaf in a cannabis flower grinder. The resulting blend of approximately 50:50 cannabis:infused flower was used to prepare thirty 0.5 g pre-rolls using a standard hand-held cigarette rolling machine. These pre-rolls were labelled A.

Next, 15 g of cannabis flower was ground in a cannabis flower grinder and the resultant ground flower was used to prepare thirty 0.5 g pre-rolls using a standard hand-held cigarette rolling machine. These pre-rolls were labelled B.

### Study

A group of 24 volunteers were recruited, 13 male and 11 female. They were all cannabis smokers, most having smoked for more than 5 years and consuming regularly at least a few times a week.

On the first test day, volunteers were given one of the two pre-rolls each to smoke. An hour later, they responded to a questionnaire on their experience. The following day, the volunteers returned and smoked the other pre-roll and completed the questionnaire as per the first day. Then they were interviewed and asked to compare the two pre-rolls on various criteria.

Overall, 85% of participants reported preferring pre-roll A. Ninety-six percent reported a positive or very positive experience with pre-roll A. Ninety-two percent said pre-roll A burned evenly or very evenly. Eight-eight percent reported that pre-roll A had a good or very good flavor. Eighty-three percent reported that pre-roll A felt smooth or very smooth to smoke. Finally, 58% reported that they got more high than they expected to with pre-roll A, 28% as expected and only 13% less than expected, even though they thought they were smoking a pure cannabis pre-roll where in fact it contained only half cannabis.

### Example 3

### Materials

Cannabis flower: Jack Herer reported to contain 24% THC. Infused leaf: contained 85% dried, precut and sieved marshmallow leaf (Althaea officinalis L); 15% CBG; 5% terpenes. Terpenes comprised a Superlemon Haze flavor. CBG was a hemp extracted isolate containing 95% CBG by weight.

Approximately 5 g of cannabis flower was blended with approximately 5 g of infused marshmallow leaf in a cannabis flower grinder. The resulting blend of approximately 50:50 cannabis:infused flower was used to prepare twenty 0.5 g pre-rolls using a standard hand-held cigarette rolling machine. These pre-rolls were labelled A.

Next, 5 g of cannabis flower was blended with 5 g of pure marshmallow leaf (no infused terpenes or CBG) in a cannabis flower grinder. The resulting blend of 50:50 cannabis:marshmallow leaf was used to prepare twenty 0.5 g pre-rolls using a standard hand-held cigarette rolling machine. These pre-rolls were labelled B.

### Study

A group of 13 volunteers were recruited, 10 male and 3 female. They all reported to regularly suffer from anxiety when consuming cannabis. Reported symptoms of anxiety when consuming cannabis included increased heart rate, racing thoughts (e.g., overthinking), and sweating (e.g., sweaty palms). They were generally less regular users.

On the first test day, volunteers were each given one pre-roll A to smoke. An hour later, they responded to a questionnaire on their experience. The following day, the volunteers returned and smoked pre-roll B and completed the questionnaire as per the first day. Then they were interviewed and asked to compare the two pre-rolls on various criteria.

Eighty-five percent of participants reported experiencing less anxiety with pre-roll A cf. B. Indeed, eight participants reported no anxiety at all with pre-roll A, with the remainder experienced only a little. Of the five reporting a little anxiety with pre-roll A, 4 experienced some anxiety with both pre-rolls yet all four reported preferring pre-roll A. In contrast, nine people experienced anxiety with pre-roll B. Of the eight reporting no anxiety with pre-roll A, three reported no anxiety with either pre-roll yet all three reported preferring pre-roll A. Only one person experienced anxiety with pre-roll A and not B, whereas 5 people experienced anxiety with pre-roll B and not A. Ninety-two percent of participants preferred pre-roll A overall, 85% found it more mellow, and 92% found it a more happy experience cf. pre-roll B.

1. A composition comprising tetrahydrocannabinol (THC) or tetrahydrocannabinolic acid (THCA) and cannabigerol (CBG) or cannabigerolic acid (CBGA) in a weight ratio THC or THCA:CBG or CBGA ranging from about 1:10 to about 10:1.

2. The composition of claim 1, wherein the composition is formulated as a liquid, a waxy material, a gel, or a solid.

3. The composition of claim 1, wherein the composition further comprises at least one terpene, wherein the composition comprises less than or equal to 10% by weight terpenes, based on the total weight of the composition, and wherein the at least one terpene is chosen from α-pinene, β-pinene, limonene, β-myrcene, β-caryophyllene, linalool, valencene, humulene, myrcene, bisabolol, geraniol, humulene, farnesene, fenchol, terpineol, borneol, nerolidol, (-)-caryophyllene oxide, terpinolene, or combinations thereof.

4. (canceled)

5. The composition of claim 1, wherein the composition comprises at least 5 mg CBG or CBGA per 0.125-100 g of the composition.

6. (canceled)

7. The composition of claim 1, wherein the composition further comprises cannabidiol (CBD), cannabichromene (CBC), cannabicyclol (CBL), cannabivarin (CBV), cannabielsoin (CBE), cannabicitran (CBT), cannabigerovarin (CBGV), tetrahydrocannabivarin (THCV), cannnabidivarin (CBDV), cannabinol (CBN), acidic forms thereof, or any combination thereof.

8. The composition of claim 7, wherein the composition comprises less than 1% by weight CBD or acidic form thereof, based on the total weight of the composition.

9. The composition of claim 1, wherein the composition is formulated as a smokeable vaping liquid, a smokeable waxy material, or a smokeable herbal material; and/or wherein the composition (a) does not comprise marijuana, (b) does not comprise hemp, or (c) does not comprise marijuana or hemp.

10-12. (canceled)

13. A method of reducing the risk of THC-induced anxiety and/or producing a different intoxicating effect compared to THC alone, the method comprising ingesting and/or inhaling the composition of claim 1.

14. A method of providing the same or similar level of potency as compared to a reference composition with a greater amount of THC or THCA per gram of the composition, the method comprising ingesting and/or inhaling the composition of claim 1.

15-17. (canceled)

18. A method of altering an intoxicating experience of cannabis or tetrahydrocannabinol (THC), the method comprising inhaling smoke or vapor from a smokeable material, wherein the smokeable material comprises:
cannabis or other THC-containing material; and
a composition comprising one or more smokeable herbs that are not marijuana and one or more cannabinoids that are not tetrahydrocannabinol (THC) or tetrahydrocannabinolic acid (THCA), wherein the one or more cannabinoids includes cannabigerol (CBG) or cannabigerolic acid (CBGA), and wherein the composition comprises less than 0.3% by weight THC and THCA, based on the total weight of the composition.

19-20. (canceled)

21. The method of claim 18, wherein the composition further comprises one or more terpenes chosen from α-pinene, β-pinene, limonene, β-myrcene, β-caryophyllene, linalool, valencene, humulene, myrcene, bisabolol, geraniol, humulene, farnesene, fenchol, terpineol, borneol, nerolidol, (-)-caryophyllene oxide, terpinolene, and combinations thereof.

22. The method of claim 18, wherein the one or more cannabinoids that are not THC or THCA further includes cannabidiol (CBD), cannabichromene (CBC), cannabicyclol (CBL), cannabivarin (CBV), cannabielsoin (CBE), cannabicitran (CBT), cannabigerovarin (CBGV), tetrahydrocannabivarin (THCV), cannnabidivarin (CBDV), cannabinol (CBN), acidic forms thereof, or any combination thereof.

23. (canceled)

24. A composition comprising:
0-10% by weight terpenes;
2-40% by weight cannabinoids that are not tetrahydrocannabinol (THC) or tetrahydrocannabinolic acid (THCA), wherein the cannabinoids that are not TCH or THCA includes cannabigerol (CBG) or cannabigerolic acid (CBGA); and
50-98% by weight of at least one smokeable herb that is not marijuana;
wherein the composition comprises less than 0.3% by weight THC and THCA, based on the total weight of the composition.

25. The composition of claim 24, wherein the composition comprises at least 5 mg of the cannabinoids that are not THC or THCA per 0.125-100 g of the composition.

26. (canceled)

27. The composition of claim 24, wherein the cannabinoids that are not THC or THCA further includes include one or more of cannabidiol (CBD), cannabichromene (CBC), cannabicyclol (CBL), cannabivarin (CBV), cannabielsoin (CBE), cannabicitran (CBT),), cannabigerovarin (CBGV), tetrahydrocannabivarin (THCV), cannnabidivarin (CBDV), cannabinol (CBN), acidic forms thereof, or any combination thereof.

28. The composition of claim 27, wherein the composition comprises less than 1% by weight CBD or acidic form thereof, based on the total weight of the composition.

29. The composition of claim 24, wherein the smokeable herb comprises Althaea officinalis L, Amaranthus dubius, Arctostaphylos uva-ursi, Argemone mexicana, Arnica, Artemisia vulgaris, Asteraceae, Brassica oleracea, Calea zacatechichi, Canavalia maritima, Cecropia obtusifolia, Cestrum nocturnum, Cynoglossum virginianum, Cytisus scoparius, Entada rheedii, Eschscholzia californica, Fittonia albivenis, Humulus japonicus, Lavandula, Lactuca virosa, Lamiaceae, Salvia, Scutellaria, Leonotis leonurus, Leonurus cardiaca, Leonurus sibiricus, Lobelia cardinalis, Lobelia inflata, Lobelia siphilitica, Nepeta cataria, Nicotiana, Nymphaea alba, Nymphaea caerulea, Papaver somnferum, Origanum majorana, Origanum vulgare, Passiflora incarnata, Pedicularis densiflora, Pedicularis groenlandica, Rubus idaeus, Rubus occidentalis, Salvia divinorum, Salvia dorrii, Scutellaria galericulata, Scutellaria lateriflora, Scutellaria nana, Sida acuta, Sida rhombifolia, Silene capensis, Syzygium aromaticum, Tagetes lucida, Tarchonanthus camphoratus, Turnera diffusa, Tussilago farfara, Verbascum, Zornia latifolia, or mixtures thereof.

30. The composition of claim 24, wherein the composition further comprises at least one terpene chosen from α-pinene, β-pinene, limonene, β-myrcene, β-caryophyllene, linalool, valencene, humulene, myrcene, bisabolol, geraniol, humulene, farnesene, fenchol, terpineol, borneol, nerolidol, (-)-caryophyllene oxide, terpinolene, or combinations thereof, and wherein the composition comprises less than or equal to 10% by weight terpenes, based on the total weight of the composition.

31-33. (canceled)

34. A method of preparing a smokeable material, the method comprising combining the composition of claim 24 with THC, cannabis, or other THC-containing material.

35. (canceled)

36. A method of reducing the risk of THC-induced anxiety and/or producing a different intoxicating effect compared to THC alone, the method comprising inhaling smoke or vapor from a smokeable material or administering the smokeable material to a subject, wherein the smokeable material comprises the composition of claim 24 and THC, cannabis, or other THC-containing material.
